(19) United States
(12) Patent Application Publication
(10) Pub. No.: US 2008/0166436 A1
(43) Pub. Date: Jul. 10, 2008

(54) Composition for normalizing otitis externa in mammals

(75) Inventor: Gary Pusillo, Marshalltown, IA (US)

Correspondence Address: HARRIS BEACH PLLC, 99 GARNSEY ROAD, PITTSFORD, NY, 14534, US

(73) Assignee: Apperon Inc., Toronto (CA)

(21) Appl. No.: 11/651,745

(22) Filed: Jan. 10, 2007

(51) Int. Cl. A61K 36/752; A61P 27/16

(52) U.S. Cl. 424/736

(57) ABSTRACT

Otitis externa is a condition of the outer ear canal in mammalian animals, especially dogs and cats, marked by inflammation and infection of tissue, buildup of ear wax, and draining of ear exudate. Symptoms of infection are, among others, excessive cerumen, alopecia in the ear flap, crusts, and ear pruritis. Typical causes of otitis externa are fungi, bacteria, and mites. A medicating mixture in accordance with the invention, comprising cold-pressed citrus oil, deionized water, and, optionally, hydrolysates of milk proteins, when applied topically to an infected ear canal can produce significant reduction in symptoms. In a clinical study, 95% of an infected population of dogs were normalized without other or further treatment.

### FIELD OF THE INVENTION

The present invention relates to topical medications for skin infections; more particularly to outer ear infections; and most particularly to an ear medicating composition for treating otitis externa comprising cold-pressed citrus oil, deionized water, and, optionally, colostrum-derived hydrolysates of milk proteins including cytokines.

### BACKGROUND OF THE INVENTION

Otitis externa is a general term applied to an infected and inflamed condition of the outer ear canal and flap of an animal. This condition is well known in various mammalian species, and especially in domestic dogs and cats. One study has found that of dogs examined routinely at veterinary clinics in Brazil, otitis externa was diagnosed in more than 20% of all patient visits.

Mites, bacteria, and fungi are agents at the root cause of otitis externa. Although any of these three organisms can be clinically managed individually, prior art medications that address any one of these agents specifically can actually promote growth of the other otitis-producing organisms. Consequently, most persistent ear infections have been found to be caused by more than one agent.

The very nature of the dog or cat ear predisposes it to a long list of problems from foreign bodies, bacteria, yeast, fungi, and parasites. The long ear canal amplifies sound but it can also be a warm, moist incubator for various types of infections.

The ears are responsible for hearing and balance. The ear flaps and outer ear canals capture and convey sound to the middle and inner ears. Long ear canals protect the important structures of the middle and inner ear from injury, but are prone to accumulations of wax and debris. Some breeds that have hair growing down the ear canal have amplified problems associated with non differentiated ear problems. Some of these breeds are Schnauzers, West Highland White Terriers, and Komondors.

Normalization of the external ear canal is essential in the prevention of serious and sometimes life-threatening problems. Middle- and inner-ear concerns are usually caused by the spread of infection, either from the external ear canal or up the Eustachian tube from the back of the throat.

Inflammation of parasitic, bacterial, or unknown cause of the deep part of the ear canal is difficult to treat. It is therefore imperative for the owners of dogs and cats to be proactive against ear related problems previously mentioned. Continued inflammation can lead to permanent changes such as ear canal cartilage calcification, fibrosis, and occlusion of the ear canal. Simple debris accumulation, excess “ear wax” (cerumen), parasites, and microbes can lead to excessive itching which can cause the animal to shake its head with such violence that certain structures of the ear can be damaged causing a hematoma that can require surgical intervention.

It is known in the prior art to treat otitis externa with various medications. The majority of ear cleaners on the market do a fairly good job of cleaning the outer ear (also called the earflap or pinna), not however through some magic ingredient or formulation, but by simply making the pet's owner physically rub away the dirt and debris once the “ear cleaner” is applied. Water and/or soap and water can accomplish the same task as most of the products currently sold in the marketplace as “pet ear cleaners.” However, it is known in the veterinary arts that one should never use soap and water to clean a dog's ears, since soapy water can cause an ear infection. Thus a dog or cat owner is in need of a safe and efficient ear cleaner whose formulation provides more than a pacifier effect.

What is needed in the art is an ear normalizing composition that can alleviate otitis externa in animals when caused by a plurality of agents, acting singly or in combination, selected from the group consisting of fungus, bacteria, and mites.

It is a principal object of the invention to alleviate symptoms of otitis externa in animals.

It is a further object of the invention to normalize a large percentage of those animals treated for otitis externa.

### BRIEF DESCRIPTION OF THE INVENTION

Briefly described, an ear cleaning composition for normalizing animals exhibiting symptoms of otitis externa comprises citrus oil, deionized water, and optionally but preferably colostrum-derived milk protein hydrolysates, preferably including cytokines.

An ear “normalizer” is an applied agent that can bring about the desired nature of the ear and its external structures and that allows the realization of the ear's genetically predetermined normal appearance and function. Normal ear characteristics include all commonly held scientific clinical observations related to the normal ear, including color of tissues, integrity of the epithelial surface, odor, cerumen content, absence of obvious itching, and funtional integrity. A normalizer allows the removal (wash, flush, rinse) of undesired debris and common unwanted invaders (bacteria, viruses, yeast, fungi, mites etc.) that typically cause abnormal ear presentations, observations, and functions. In addition the a normalizer alleviates, diminishes, and reverses some or all of the effects of ear debris and invaders, resulting in normalized clinical observations being accomplished without the harmful effects on healthy tissue typically seen in prior art.

The composition may be used as a direct application or applied via a soft gauze or cotton pad. In most cases of otitis externa, moistening of the canal for cats requires 0.5-1.0 cc and for dogs 1.0-5.0 cc applied to all areas of the ear and ear canal, followed by a gentle cleaning to enable deep cleaning action of the ear cleaner in removing unwanted material from deep within the ear canal. Applications generally are repeated at one-week intervals for up to four weeks, although yeast infections can require a cleaning every three days until the infection is gone or is totally under control.

### DETAILED DESCRIPTION OF THE INVENTION

It has been found that cold-pressed citrus oil, derived by pressing without heat in known fashion from the rind of any of various citrus fruits, is highly effective in alleviating and normalizing the symptoms of otitis externa.

In a first embodiment of a normalizing ear cleaner composition in accordance with the invention, the citrus oil is diluted with deionized water to a level of about 65%-85% citrus oil and about 15%-35% deionized water; and preferably to a level of about 78% citrus oil and 22% deionized water, as described in more detail below.

Preferably, the citrus oil of the invention is an orange oil derived from Citrus aurantium and/or Citrus reticulata by cold pressing, although citrus oils derived from other citrus fruits are also contemplated within the scope of the invention. This process is an important aspect of providing citrus oils that can perform the intended functions of the present invention. By preventing the accumulation of D' Limonene, as is known to be found in citrus-based oils produced by processes such as distillation or solvent extraction, the cold-press process used to obtain the citrus oil used in the present normalizing ear cleaner composition does not contain an aggressive orange distillate that is known to cause tissue irritation.

In the present cold-press process, the citrus oils are concentrated to a point just short of gellation. The residual moisture level of the oils allows them to be flowable under most conditions commonly found in homes, on store shelves, in veterinary clinics, and during transportation. Typically, the range in water content of the component referred to in the formulation as “cold-pressed citrus oil” in accordance with the invention is between about 68% and about 76%. This component preferably also contains surfactants, stabilizers, preservatives, a bactericide, colorants, a pH adjuster, an inorganic base, and sodium tetraborohydrate decahydrate. Surfactants form preferably about 21% of this component prior to the addition of deionized water.

The preferred formulation of the first embodiment composition is 77.42% “cold-pressed citrus oil” as defined above and 22.58% deionized water.

In a second embodiment of a normalizing ear cleaner composition in accordance with the invention, citrus oil is diluted with deionized water to a level of about 78% citrus oil and the mixture is fortified with colostrum-derived proteins to a level of about 3.2%. The preferred formulation of the second embodiment therefore is 77.42% “cold-pressed citrus oil”, 19.35% deionized water, and 3.23% colostrum-derived proteins including cytokines.

The colostrum-derived proteins include a variety of hormones, enzymes, and cytokines that are highly useful in normalizing inflammation and infection in the ear canal.

As with any surface integrity challenge, the surface lining (epithelial tissue) of the ear canal can be compromised in numerous ways resulting in inflammation. Typically, inflammation is the response to the presence of microorganisms or to injury. Inflammation is a vital protective mechanism since it is the means by which phagocytic cells and defensive molecules such as antibodies gain access to sites of microbial invasion or tissue damage. The cytokine blend that is supplied in the ear cleaner, derived preferably from bovine colostrum, is included to help the body's innate immunity and to promote tissue healing by reducing damage and enhancing the repair of damaged tissue. The cytokines in the ear cleaner composition are very versatile and flexible in their activity at the surface area in the ear canal. Cytokines are low molecular weight regulatory proteins secreted from white blood cells (lymphocytes) as well as a variety of other cells in response to inducing stimuli. Cytokines typically are recovered from colostrum, for example, bovine colostrum. Some cytokines have singular roles while other have multiple activities. Cytokines include Interferon (named for its interference with viral replication), Migration Inhibition Factor (MIF), Lymphotoxin (LT), Tumor Necrosis Factor (TNF), Interleukins, Chemokines, several growth factors, and lymphokines. Cytokines share many properties with hormones and growth factors and the distinction in the art between these three classes of mediators is becoming very blurry. In the present invention, cytokines regulate the intensity and duration of the immune response by stimulating or inhibiting the activation, proliferation, and/or differentiation of various cells, and by regulating the secretions of antibodies or other cytokines.

With many ear problems related to allergic reactions, cytokine support is an essential element in modulating the negative results of such a situation in the ear canal. Many organisms are associated with an animal's body surface, and thus any compromise in the integrity of the epithial lining of the ear canal can present an opportunity for the tissues to be invaded and damaged. These invasions can result in allergic reactions due to the body's acquired immune system's ability to defend itself against invading microorganisms or those substances that are recognized as exogenous antigens.

In a third embodiment of an ear cleaner composition in accordance with the invention, cold-pressed citrus oils are partially replaced by a class of compounds referred to herein as “active oils”. Examples of such active oils are Buriti oil, Andiroba, Babacu, Castanha do Brasil, Passiflora Edulis seed oil, Passiflora Incarnata Seed Oil, Pequi oil, Acai Oil, Annato oil, Brazil nut oil, and Copaiba oil. Such oils have various specific normalizing properties that can be of great advantage in some cases of otitis externa.

In a method for use of a normalizing ear cleaner in accordance with the invention, the composition can be applied directly via dropper or indirectly via a soft gauze or cotton pad. It is important to allow the cleaner to migrate into the ear canal so as to get next to the tympanic membrane. It is also vitally important to be certain that there is a tympanic membrane before introducing the medication into an ear. After a proper application of the composition, the ear “wax” and accompanying unwanted materials will flake up and will be easily groomed away by the animal's natural grooming process, or alternatively the flakes can be wiped out of the ear with a soft cotton or gauze pad. In most cases of otitis externa, moistening of the ear canal for cats requires ½ cc -1 cc and dogs 1-5 cc of the composition applied to all areas of the ear and ear canal, followed by a gentle cleaning with soft cotton or gauze pads, to enable the deep cleaning action of the ear cleaner composition to remove the unwanted materials from deep within the canal.

After the first cleaning and the flaking of the foreign matter, application of the composition is repeated preferably only once per week until the ear becomes normal. Treatment should be repeated weekly until desired results are accomplished. Yeast infections may require a cleaning every three days until the infection is gone or totally under control. Usually large ears will require 1 cc of composition in cats and 5 cc in dogs. If the hair surrounding the external ear canal is soiled with exudates, it is advisable to shave the hair if possible. If shaving is impractical, then saturating the soiled hair with the normalizing ear cleaner is advisable so as to allow the cleaner an opportunity to suppress bacteria and yeast growth.

In cases of otitis externa wherein infection and inflammation are minimal and the principal symptom is excess cerumen and dirt, the first embodiment of an ear cleaner composition described above may be all that is required for loosening and flushing of matter to achieve ear normalization. For more serious cases involving extensive infection, rupture of the skin, and inflammation, the second or third embodiments described above are preferred treatments.

From the foregoing description, it will be apparent that there has been provided an improved normalizing ear cleaner for alleviating otitis externa in animals, and especially in cats and dogs. Variations and modifications of the herein described normalizing ear cleaner, in accordance with the invention, will undoubtedly suggest themselves to those skilled in this art. Accordingly, the foregoing description should be taken as illustrative and not in a limiting sense.

What is claimed is:

1. An ear cleaner composition for normalizing otitis externa in mammals, comprising:
a) cold-pressed citrus oils; and
b) deionized water.

2. An ear cleaner composition in accordance with claim 1 wherein said cold-pressed citrus oils are derived from the group consisting of Citrus aurantium, Citrus reticulata, and combinations thereof.

3. An ear cleaner composition in accordance with claim 1 wherein said cold-pressed citrus oils further comprise materials selected from the group consisting of water, surfactants, stabilizers, preservatives, bactericide, colorants, pH adjuster, inorganic base, sodium tetraborohydrate decahydrate, and combinations thereof.

4. An ear cleaner composition in accordance with claim 1 wherein said cold-pressed citrus oils are present in an amount between about 65% and about 85%, and wherein said deionized water is present in an amount between about 15% and about 35%.

5. An ear cleaner composition in accordance with claim 4 wherein said cold-pressed citrus oils are present at 77.42%, and wherein said deionized water is present at 22.58%.

6. An ear cleaner composition in accordance with claim 1 further comprising colostrum-derived proteins.

7. An ear cleaner composition in accordance with claim 6 wherein said colostrum-derived proteins are bovine.

8. An ear cleaner composition in accordance with claim 6 wherein said colostrum-derived proteins include cytokines.

9. An ear cleaner composition in accordance with claim 6 wherein said cold-pressed citrus oils are present in an amount between about 65% and about 85%, wherein said deionized water is present in an amount between about 15% and about 35%, and wherein said colostrum-derived proteins are present in an amount between about 2% and about 5%.

10. An ear cleaner composition in accordance with claim 9 wherein said cold-pressed citrus oils are present at 77.42%, wherein said deionized water is present at 19.35%, and wherein said colostrum-derived proteins are present at 3.23%.

11. An ear cleaner composition in accordance with claim 1 further comprising active oils selected from the group consisting of Buriti oil, Andiroba, Babacu, Castanha do Brasil, Passiflora Edulis seed oil, Passiflora Incarnata Seed Oil, Pequi oil, Acai Oil, Annato oil, Brazil nut oil, Copaiba oil, and combinations thereof.

12. (canceled)

13. A method for normalizing otits externa in the ear canal of an animal, comprising the steps of:
a) providing an ear cleaner composition including cold-pressed citrus oils and deionized water;
b) applying a dose of said ear cleaner composition to the skin and hair surfaces of said animal ear canal;
c) removing loosened debris from said skin and hair; and
d) repeating steps b) and c) at weekly intervals as needed until said ear is normalized.

14. A method in accordance with claim 13 comprising the further step, before said applying step, of confirming the presence of a tympanic membrane in said ear.

15. A method in accordance with claim 13 wherein said ear cleaner composition further includes colostrum-derived proteins.

16. A method in accordance with claim 13 wherein said dose is between about 0.5 cc and about 1.0 cc for cats and between about 1.0 cc and about 5.0 cc for dogs.
